(19) United States
(12) Patent Application Publication
(10) Pub. No.: US 2020/0138449 A1
(43) Pub. Date: May 7, 2020

(54) SYSTEM AND METHOD FOR BLENDING MEDICAL GASES

(71) Applicant: Roger S. Hogue, Maple Grove, MN (US)

(72) Inventor: Roger S. Hogue, Maple Grove, MN (US)

(21) Appl. No.: 16/675,818

(22) Filed: Nov. 6, 2019

Related U.S. Application Data
(60) Provisional application No. 62/756,438, filed on Nov. 6, 2018.

(51) Int. Cl. A61B 17/12; A61M 5/19

(52) U.S. Cl. CPC A61B 17/12186; A61M 2005/006; A61B 2017/1205; A61M 5/19

(57) ABSTRACT

A medical gas blending system includes a first syringe configured to be connectable to a supply of a first gas, and a second syringe configured to be connectable to a supply of a second gas. A three-way valve is provided between the first syringe and the second syringe. The three-way valve is operable in a first configuration to selectively isolate the first syringe from the second syringe, in a second configuration to allow communication between the first syringe and the second syringe, so that the first gas present in the first syringe and the second gas present in the second syringe may be selectively blended in the first syringe and/or the second syringe to form a blended gas mixture, and in a third configuration so that the blended gas mixture may be transferred from the first syringe and/or the second syringe to an external syringe connectable to the three-way valve.

### CROSS-REFERENCE TO RELATED APPLICATION(S)

This application claims the benefit of U.S. Provisional Application No. 62/756,438 filed on Nov. 6, 2019, which is incorporated by reference herein in its entirety.

### BACKGROUND

The present invention relates to a low-pressure, portable system and method for blending and delivering medical gases that allows custom gas blends to be formed from gases stored in supply cylinders, for use in medical applications that use either a single gas or a gas mixture.

Some medical procedures, such as sclerotherapy, for example, may be performed using foam that is made by mixing air or a gas mixture with a solution (e.g., a sclerosant solution). In these procedures, it can be beneficial to control the proportion of gases in the gas mixture, so that the foam has a preferred physiologic gas composition. In one particular example, for foam sclerotherapy, a gas mixture of 65% O₂ and 35% CO₂ may be mixed with a polidocanol liquid sclerosant solution to form a therapeutic injectable foam. Other proportions of O₂ and CO₂ gases may be used in other examples.

Presently, medical gas mixtures may be purchased and stored with a fixed, specified gas ratio; for example, a canister may be purchased that is a 70:30 O₂—CO₂ mixture. However, this is an expensive and inefficient way to purchase and stored medical gases, particularly in clinic environments where the supplies of the constituent gases are (or could be) present in their individual form.

### SUMMARY

A medical gas blending system includes a first syringe configured to be connectable to a supply of a first gas, and a second syringe configured to be connectable to a supply of a second gas. A three-way valve is provided between the first syringe and the second syringe. The three-way valve is operable in a first configuration to selectively isolate the first syringe from the second syringe, in a second configuration to allow communication between the first syringe and the second syringe, so that the first gas present in the first syringe and the second gas present in the second syringe may be selectively blended in the first syringe and/or the second syringe to form a blended gas mixture, and in a third configuration so that the blended gas mixture may be transferred from the first syringe and/or the second syringe to an external syringe connectable to the three-way valve.

### BRIEF DESCRIPTION OF THE DRAWINGS

FIG. 1 is an illustration of a two-syringe medical gas blending system according to an embodiment of the present disclosure.

FIG. 2 is an illustration showing the two-syringe medical gas blending system of FIG. 1 connected to gas supply containers.

FIGS. 3 and 4 are illustrations of various features and embodiments of medical gas blending systems.

FIGS. 5A-5E are illustrations of another exemplary configuration of a medical gas blending system according to an embodiment of the present disclosure.

### DETAILED DESCRIPTION

Disclosed herein are a portable system and method for creating a custom medical gas mixture of a physician's choice using a syringe filling system that connects to metal medical gas cylinders (e.g., steel or aluminum).

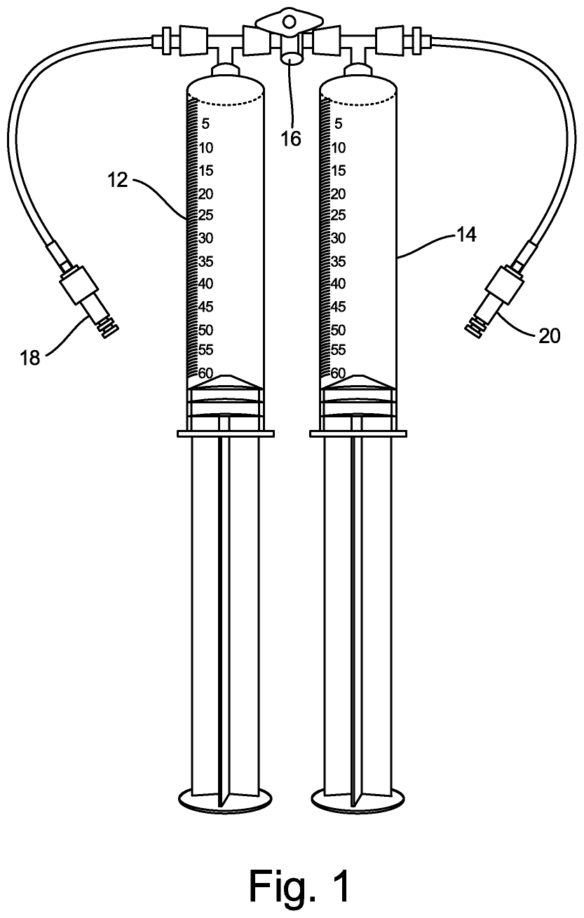

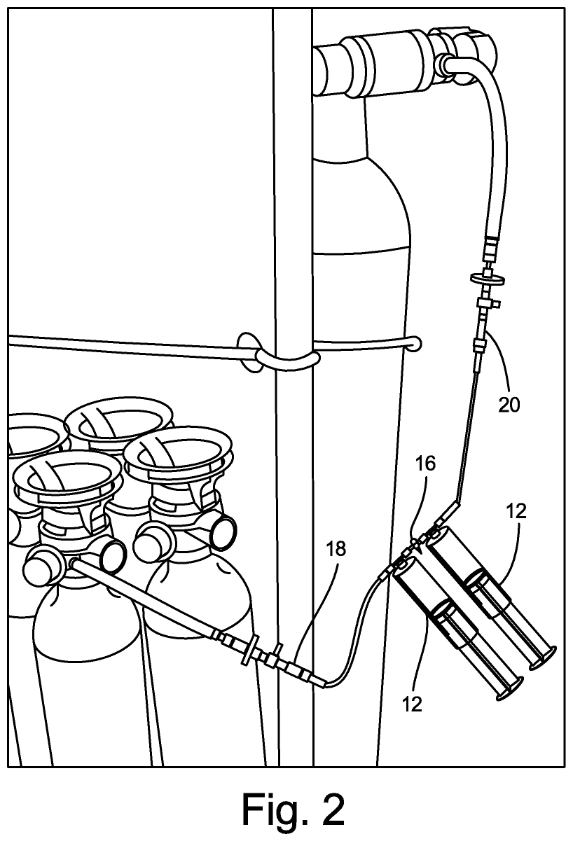

FIG. 1 is an illustration of two-syringe medical gas blending system 10 according to an embodiment of the present disclosure. System 10 includes first syringe 12 (for example, for O₂ gas), second syringe 14 (for example, for CO₂ gas), three-way luer stopcock valve 16, fitting 18 (for example, to connect to an O₂ gas supply container), and fitting 20 (for example, to connect to a CO₂ gas supply container). FIG. 2 is an illustration showing two-syringe medical gas blending system 10 connected to an O₂ gas supply container (on the side of first syringe 12) and connected to a CO₂ gas supply container (on the side of second syringe 14).

In operation, system 10 allows two medical gases (or more, in some embodiments) contained within medical gas supply containers to be individually filled into the first and second syringes 12, 14. First syringe 12 and second syringe 14 are isolated and separated from one another by one-way luer stopcock valve 16, and the gases in first syringe 12 and second syringe 14 may then be mixed together after selected gas ratios are chosen based on their respective volumes. Specifically, opening one-way luer stopcock valve 16 between first syringe 12 and second syringe 14 allows combining of the selected medical gases into one of the two syringes, by depressing the plunger in one syringe to force the gas in that syringe into the other syringe, thereby forming a mixture of the two gases. This gas mixture may be created by a physician at a patient's bedside, without the need to purchase a pre-mixed medical gas mixture from a medical gas supplier.

For example, a physician may connect fitting 18 to an O₂ supply cylinder, and may connect fitting 20 to a CO₂ supply cylinder, as shown in FIG. 2. Stopcock valve 16 is initially kept in a closed position, to block the path for gas to travel between first syringe 12 and second syringe 14. The physician may then turn on the gas supply from the O₂ supply cylinder, and release the plunger of first syringe 12 to allow 32.5 ml of O₂ gas to fill first syringe 12. The gas supply from the CO₂ supply cylinder may then be turned on, and the plunger of second syringe 14 may be released to allow 17.5 ml of CO₂ gas to fill second syringe 14. Then, stopcock valve 16 may be turned to an open position, to allow communication between first syringe 12 and second syringe 14. The physician may then release the plunger of either first syringe 12 or second syringe 14, and depress the plunger of the other of first syringe 12 or second syringe 14, so that 50 ml of a mix of 65% O₂ and 35% CO₂ is present in one of the syringes. A separate, sterile syringe intended to interface clinically with a patient, typically having a separate luer-connection and valve or stopcock, may then be used to draw a desired volume of the gas mixture for use in a medical procedure, such as to produce a sclerosant foam, for example.

Analysis and verification of a prepared O2:CO2 (e.g., 65%:35%) medical gas mixture can be achieved using a gas analyzer, such as a headspace-type analyzer like the Quantek Model 902D analyzer. With a gas analyzer of this type, it is possible to take a sample (for example, about 40 cubic centimeters (ccs)) of the gas mixture to determine the proportion of oxygen and carbon dioxide within the gas mixture for quality control purposes. In other embodiments, or for other applications, different proportions of gases in the blended gas mixture may be prepared, such as 70% O₂: 30% CO₂, or any other desired proportion or ratio.

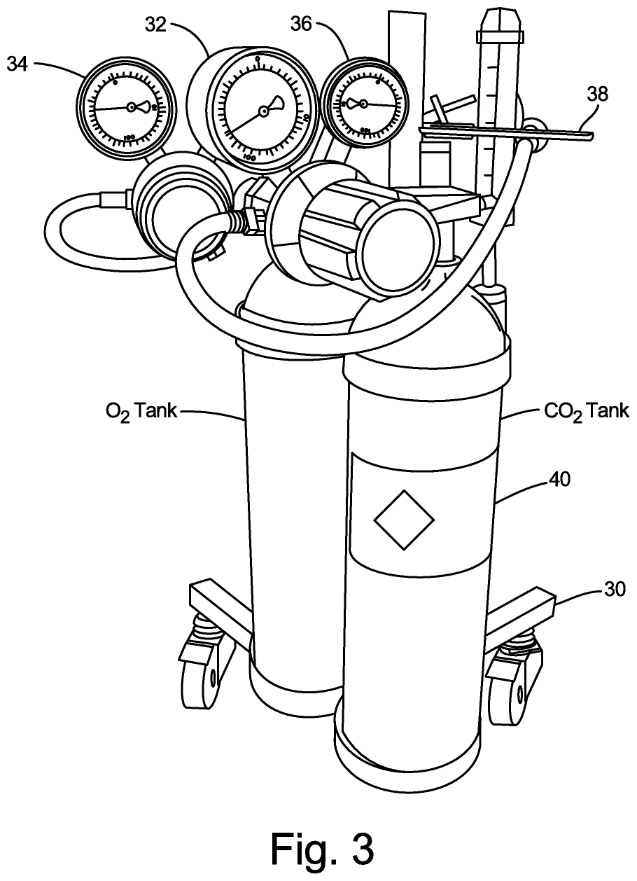

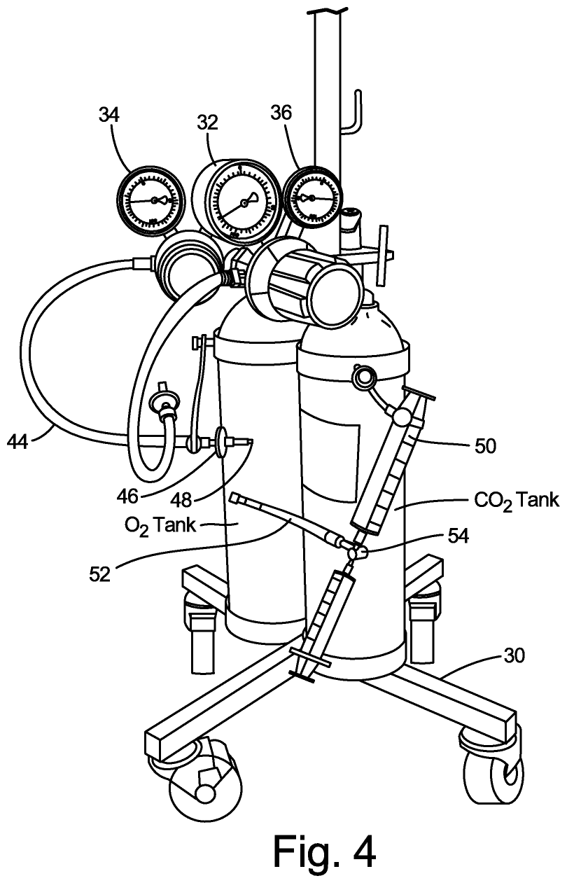

FIGS. 3 and 4 are photographs of another embodiment of a medical gas blending system. The embodiment shown in FIGS. 3 and 4 is similar to the embodiment shown in FIGS. 1 and 2, with slightly different connection configurations. The system is carried by portable cart 30, which supports an O₂ tank and a CO₂ tank in the embodiment shown. In other embodiments, additional and/or different gas supply tanks could be carried by portable cart 30. The O₂ and CO₂ tanks are equipped with high-pressure gauge 32, low-pressure (e.g., 0-10 psi) regulator gauge 34 connected to the O₂ tank, and low-pressure (e.g., 0-10 psi) regulator gauge 36 connected to the CO₂ tank. In one example, the regulator gauges may be Concoa 213 Series dual-stage regulators, or a similar model regulator, that provides the ability to deliver non-corrosive, high purity gases at low pressure with a high degree of control. Other regulation equipment could be used in other embodiments. Gas cylinder wrench 38 may also be employed with the CO₂ tank and/or with the O₂ tank. High-pressure gauge 32 depicts the gas pressure within a respective tank cylinder, which provides an indication of when the compressed gas level in the tank cylinder approaches empty. Low-pressure regulator gauges 34, 36 include controls that allow the user to precisely control the delivery pressure of gas from the respective tank cylinder, which will be discussed in more detail below. Also, the CO₂ tank and the O₂ tank are provided with medical grade gas labels 40 (affixed by the gas company that originally filled the tank, in accordance with FDA regulations), indicating the contents of the gas tanks.

As can be seen in FIG. 4, silicone tubes 44 are connected from the gas regulators to pore filter luer connections 46. In one example, the pore filter may have 0.2 micron pores. The pore filter luer connections 46 include a swabbable luer access device 48, with an accessible female luer connector. This can be connected to gas blending system 50 via silicone tube 52 having male luer fittings on each end. Gas blending system 50 includes two syringes connected by 3-way stopcock 54 at a junction between the two syringes and the silicon tube 52.

In operation, O₂ and CO₂ gas is supplied to the respective syringes of gas blending system 50 under control of regulators 34 and 36, through silicone tubes 44, pore filter luer connections 46 and luer access device 48 having silicone tube 52 connected thereto via its male luer fitting. In the embodiment shown, low-pressure regulator gauges 34, 36 include user-adjustable controls for controlling the delivery pressure of gas from the respective tank cylinder, through silicone tubes 44, pore filter luer connections 46, luer access device 48, and silicone tube 52 into gas blending system 50. In one example, the gas delivery pressure may be set by the user at 2-3 PSI for filling the syringes of gas blending system 50. Syringes with plungers that pull back to a maximum preset volume, and then are fixed or anchored from additional plunger pull back, may be beneficially used in some embodiments.

In an exemplary process for creating a gas mixture of 65% O₂ and 35% CO₂, O₂ gas may first be provided (at a controlled, low pressure) to a first syringe of gas blending system 50, by connecting silicone tube 52 to the O₂ silicone tube 44 via luer access device 48. In order for the O₂ gas to be directed to the first syringe, 3-way stopcock 54 is turned to a first position that directs the O₂ gas from silicone tube 52 to the first syringe, until a desired volume of O₂ gas fills the first syringe. Then, O₂ gas may be discontinued, and 3-way stopcock 54 may be turned to a second (or third) position that blocks any additional gas from entering or exiting the first syringe of gas blending system 50. Next, CO₂ gas may be provided (at a controlled, low pressure) to a second syringe of gas blending system 50, by connecting silicone tube 52 to the CO₂ silicone tube 44 via luer access device 48. In order for the CO₂ gas to be directed to the second syringe, 3-way stopcock 54 is turned to a third position that directs the CO₂ gas from silicone tube 52 to the second syringe, until a desired volume of CO₂ gas fills the second syringe. Then, CO₂ gas may be discontinued, and 3-way stopcock 54 may be turned to the second (or first) position that blocks any additional gas from entering or exiting gas blending system 50.

Once the first and second syringes have been filled with the desired amounts of O₂ gas and CO₂ gas, with 3-way stopcock 54 in the second position, the first and second syringes may communicate with each other, and one of the two syringes may receive the gas from the other of the two syringes, as was described above with respect to FIGS. 1 and 2.

Silicone tube 52 is connected at one end to luer access device 48, which includes a swabbable female luer connection, and at the other end to 3-way stopcock 54, which includes another swabbable female luer connection. Therefore, each end of silicone tube 52 is provided with a male luer connection, for fitting with the two swabbable female luer connections. Once the desired gas mixture is filled in one of the syringes of gas blending system 50, silicone tube 52 may be disconnected, and a separate, sterile syringe that is intended to interface clinically with a patient may be luer-connected via a male luer connection to the swabbable female luer connection of 3-way stopcock 54, to draw a desired volume of the gas mixture for use in a medical procedure, such as to produce a sclerosant foam, for example. The swabbable female luer connection of 3-way stopcock 54 may be swabbed and sterilized prior to this connection to maintain sterility of the clinical syringe. The sterile syringe is typically an assembly that includes its own valve or stopcock, to allow the gas mixture to be drawn in and retained for later use.

In some situations, it may be desirable to supply a medical gas without any blending or mixing. The system shown in FIGS. 3 and 4 can allow this, by drawing pure medical gas from the swabbable female luer access device 48 with a sterile syringe (connected by its male luer connector) intended for interfacing clinically with a patient. Therefore, the system disclosed herein provides significant flexibility for a physician to draw medical gases or blended gas mixtures for various medical procedures.

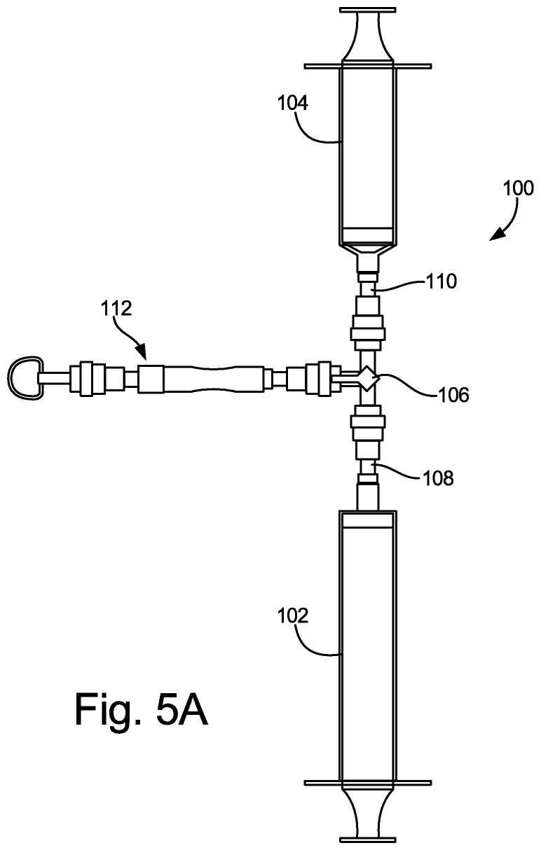

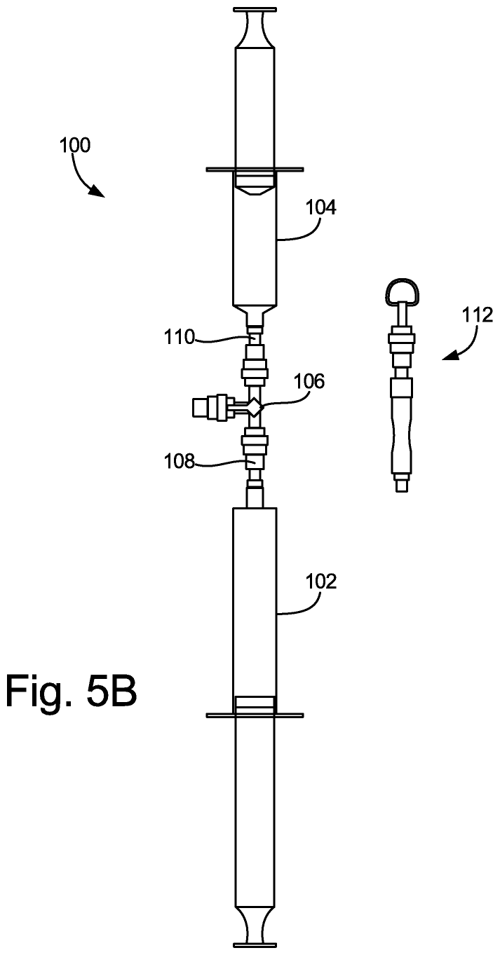

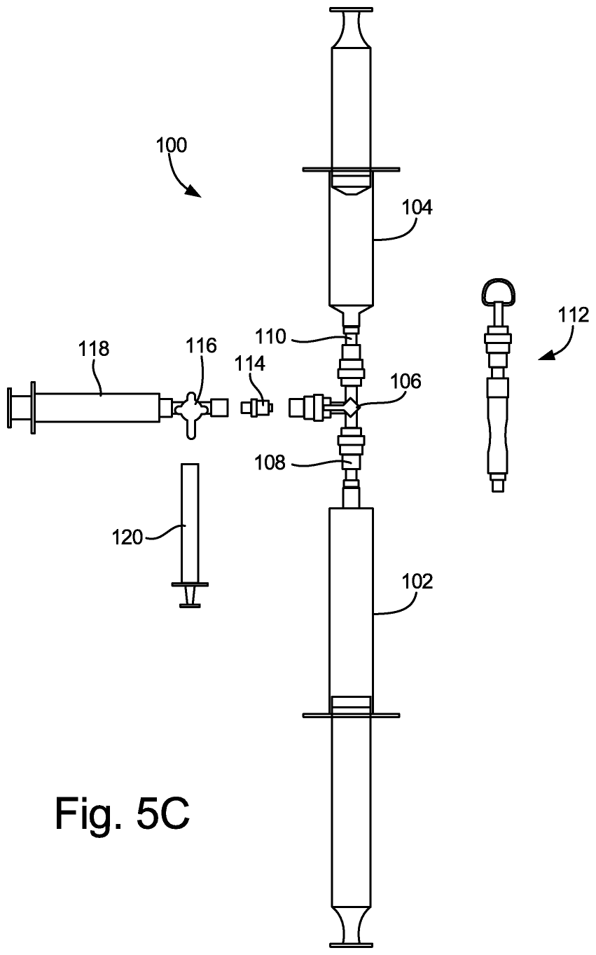

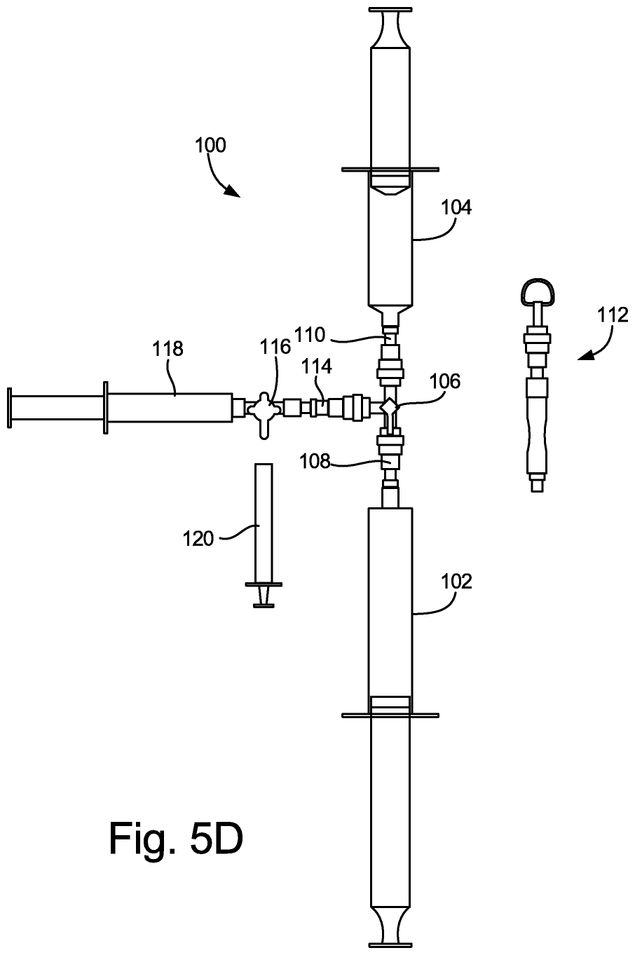

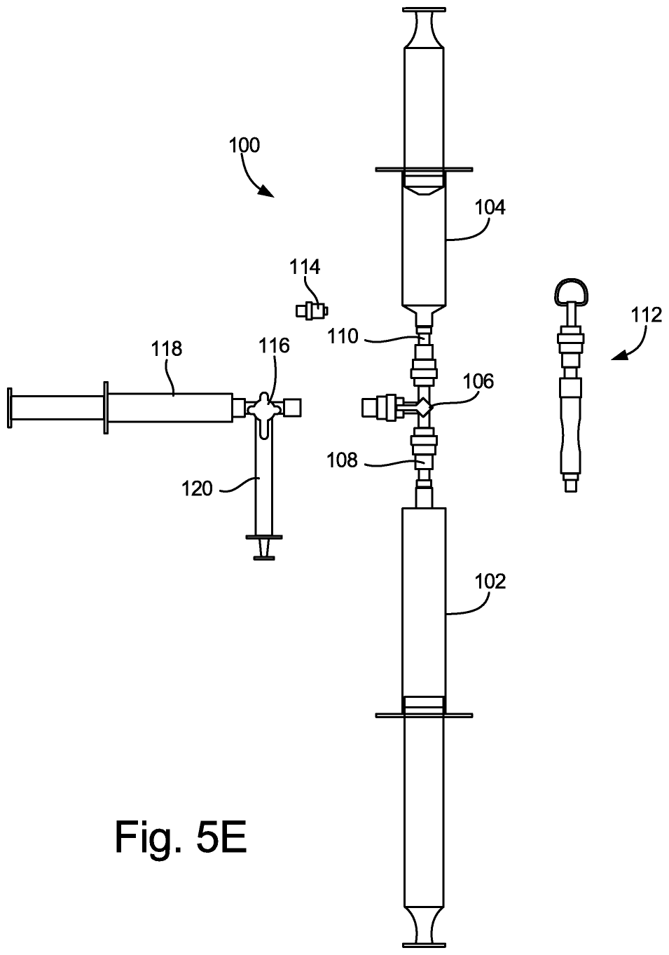

FIGS. 5A-5E illustrate an exemplary configuration of medical gas blending system 100, which is a slight variation of the configurations of system 10 shown in FIGS. 1 and 2 and of system 50 shown in FIG. 4. As shown in FIG. 5A, system 100 includes first syringe 102 (a 60 ml syringe that is shown as empty, but is configured to receive O₂ gas, for example), second syringe 104 (a 30 ml syringe that is shown as empty, but is configured to receive CO₂ gas, for example), and three-way luer stopcock valve 106. First syringe 102 is connected to female-to-male luer connector 108, which includes a pass-through filter (for example, having an array of 500 micron apertures), and which in turn is connected to a first female luer port of stopcock valve 106. Second syringe is connected to female-to-male luer connector 110, which also includes a pass-through filter (for example, having an array of approximately 500 micron (±10%) apertures), and which in turn is connected to a second female luer port of stopcock valve 106. In other embodiments, the apertures could be larger or smaller than 500 microns. The third port of stopcock valve 106 is shown in FIG. 5A as being connected to tubing/ring holder apparatus 112, which allows the system 100 to be hung up onto a hook off a gas cylinder cart when being stored. Thus, the configuration shown in FIG. 5A is ready for use, but is not yet in the process of being used.

FIG. 5B illustrates medical gas blending system 100 in a first stage of use. As shown in FIG. 5B, first syringe 102 is filled with 55.7 ml of O₂ gas (which may be done by any of the gas filling methods previously described), and second syringe 104 is filled with 30 ml of CO₂ gas (which also may be done by any of the gas filling methods previously described). Each of first syringe 102 and second syringe 104 is equipped with a plunger locks, to limit the travel of the plungers in first syringe 102 and second syringe 104 as desired. The third port of stopcock valve 106 is shown in FIG. 5B as not being connected to anything, and tubing/ring holder apparatus 112 is shown as disconnected from the third port of stopcock valve 106. Thus, the configuration shown in FIG. 5B has O₂ gas and CO₂ gas in first syringe 102 and second syringe 104 ready for blending together, and for subsequent transfer via the third port of the stopcock valve 106.

FIG. 5C illustrates medical gas blending system 100 in a second stage of use. As shown in FIG. 5C, first syringe 102 and second syringe 104 are both filled with a 65%/35% mixture of O₂ gas and CO₂ gas that have been blended together. For example, the gases may be blended together by forcefully pushing the plunger of either the first syringe 102 or the second syringe 104 after both syringes are filled with separate gases (as shown in FIG. 5B), which causes the two gases to be mixed together under pressure in the opposite syringe (with the volume in the syringe where the gases are being mixed together being maintained by the plunger lock on that syringe). Then, the plunger that had been pushed can be released back to its original position, which allows the mixed gases to fill both syringes at an equilibrium pressure. The third port of stopcock valve 106 is shown in FIG. 5C as not being connected to anything, but is ready for connection via female-to-male luer connector 114 to three-way stopcock valve 116 connected to syringe 118, which can be used to transfer the blended gas mixture to syringe 120. In some embodiments, syringe 118 and three-way stopcock valve 116 may be constructed as a single, inseparable part, while in other embodiments syringe 118 may be connectable and disconnectable from three-way stopcock valve 116, and optionally a luer fitting may be provided therebetween. Tubing/ring holder apparatus 112 is again shown as disconnected from the third port of stopcock valve 106.

FIG. 5D illustrates medical gas blending system 100 in a third stage of use. As shown in FIG. 5D, female-to-male luer connector 114 is connected to the third port of stopcock valve 106, and is also connected to a first port of three-way stopcock valve 116 that is connected (via its second port) to syringe 118. Syringe 120 is shown as not yet connected to a third port of stopcock valve 116. With these connections, a controlled volume of the blended gas mixture contained in first syringe 102 and second syringe 104 can be drawn into syringe 118. For example, as shown in FIG. 5D, approximately 8 ml of the blended gas mixture is drawn into syringe 118. This blended gas mixture is then ready for transfer to syringe 120.

FIG. 5E illustrates medical gas blending system 100 in a fourth stage of use. As shown in FIG. 5E, stopcock valve 116 (and syringe 118) is disconnected from female-to-male luer connector 114 and stopcock valve 106, as the blended gas mixture to be transferred to syringe 120 has already been stored in syringe 118. Then, a desired volume of the blended gas mixture may be transferred to syringe 120 from syringe 118 through stopcock valve 116, and used for clinical procedures, such as to produce a sclerosant foam, for example.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A medical gas blending system, comprising:
a first syringe configured to be connectable to a supply of a first gas;
a second syringe configured to be connectable to a supply of a second gas;
a three-way valve connected between the first syringe and the second syringe, the three-way valve being operable in a first configuration to selectively isolate the first syringe from the second syringe, in a second configuration to allow communication between the first syringe and the second syringe, so that the first gas present in the first syringe and the second gas present in the second syringe may be selectively blended in the first syringe and/or the second syringe to form a blended gas mixture, and in a third configuration so that the blended gas mixture may be transferred from the first syringe and/or the second syringe to a third syringe connectable to the three-way valve.

2. The medical gas blending system of claim 1, wherein the first gas comprises O₂ gas, and the second gas comprises CO₂ gas.

3. The medical gas blending system of claim 2, wherein the blended gas mixture is 65% O₂ gas and 35% CO₂ gas.

4. The medical gas blending system of claim 1, further comprising a holding apparatus connectable to the three-way valve and configured to hang the medical gas blending system for storage.

5. The medical gas blending system of claim 1, wherein the first syringe and the second syringe include plunger locks configured to limit the movement of plungers in the first syringe and the second syringe.

6. The medical gas blending system of claim 1, wherein the first syringe and the second syringe are connected to the three-way valve through filters comprising an array of apertures.

7. The medical gas blending system of claim 6, wherein the apertures have a diameter of about 500 microns.

8. The medical gas blending system of claim 1, further comprising:
a second three-way valve connectable to the first three-way valve and to the third syringe; and
a fourth syringe connected to the second three-way valve;
wherein the second three-way valve is operable in a first configuration to allow communication between the first syringe and/or the second syringe via the first three-way valve to the fourth syringe, so that the blended gas mixture in the first syringe and/or the second syringe can be transferred to the fourth syringe, and in a second configuration to allow communication between the fourth syringe and the third syringe, so that the blended gas mixture in the fourth syringe can be transferred to the third syringe.

9. The medical gas blending system of claim 8, wherein the fourth syringe and the second three-way valve are integrally connected.

10. The medical gas blending system of claim 8, wherein the fourth syringe and the second three-way valve are releasably connected.

11. A method of blending medical gases, the method comprising:
filling a first syringe with a first volume of a first gas;
filling a second syringe with a second volume of a second gas;
connecting the first syringe and the second syringe via a first three-way valve, and blending the first gas and the second gas together in the first and second syringes so that a blended gas mixture having a selected ratio of the first gas to the second gas is present in the first syringe and the second syringe;
connecting a third syringe to the first three-way valve, and transferring a selected volume of the blended gas mixture into the third syringe.

12. The method of claim 11, wherein filling the first syringe with the first volume of the first gas comprises connecting the first syringe to a supply of the first gas via a first fitting, and wherein filling the second syringe with the second volume of the second gas comprises connecting the second syringe to a supply of the second gas via a second fitting.

13. The method of claim 11, wherein the first gas comprises O₂ gas, and the second gas comprises CO₂ gas.

14. The method of claim 13, wherein the blended gas mixture is 65% O₂ gas and 35% CO₂ gas.

15. The method of claim 11, wherein connecting the first syringe and the second syringe via a first three-way valve, and blending the first gas and the second gas together in the first and second syringes so that a blended gas mixture having a selected ratio of the first gas to the second gas is present in the first syringe and the second syringe, comprises:
pushing a plunger of the first syringe to force the first gas through the first three-way valve into the second syringe under pressure to blend the first gas with the second gas to form the blended gas mixture in the second syringe, while retaining a position of a plunger of the second syringe with a plunger lock, and
pulling the plunger of the first syringe to allow the blended gas mixture to fill the first syringe and the second syringe through the first three-way valve at equilibrium pressure;
or
pushing a plunger of the second syringe to force the second gas through the first three-way valve into the first syringe under pressure to blend the second gas with the first gas to form the blended gas mixture in the first syringe, while retaining a position of a plunger of the first syringe with a plunger lock, and
pulling the plunger of the second syringe to allow the blended gas mixture to fill the second syringe and the first syringe through the first three-way valve at equilibrium pressure.

16. The method of claim 11, wherein the first syringe and the second syringe are connected to the first three-way valve through filters comprising an array of apertures.

17. The method of claim 16, wherein the apertures have a diameter of about 500 microns.

18. The method of claim 11, wherein connecting a third syringe to the first three-way valve, and transferring a selected volume of the blended gas mixture into the third syringe comprises:
connecting a second three-way valve to the first three-way valve, the second three-way valve being connected to a fourth syringe;
transferring the blended gas mixture from the first syringe and/or the second syringe through the first three-way valve and the second three-way valve to the fourth syringe;
disconnecting the second three-way valve from the first three-way valve; and
connecting the third syringe to the second three-way valve, and transferring the blended gas mixture from the fourth syringe to the third syringe through the second three-way valve.

19. The method of claim 18, wherein the fourth syringe and the second three-way valve are integrally connected.

20. The method of claim 18, wherein the fourth syringe and the second three-way valve are releasably connected.
